(19) United States
(12) Patent Application Publication
(10) Pub. No.: US 2007/0031812 A1
(43) Pub. Date: Feb. 8, 2007

(54) Processing of platelet-containing biological fluids

(75) Inventor: Stein Holme, Chino Hills, CA (US)

Correspondence Address: LEYDIG VOIT & MAYER, LTD, 700 THIRTEENTH ST. NW, SUITE 300, WASHINGTON, DC, 20005-3960, US

(73) Assignee: Pall Corporation, East Hills, NY (US)

(21) Appl. No.: 10/557,260

(22) PCT Filed: Jun. 15, 2004
(86) PCT No.: PCT/US04/19069

§ 371 Date: Nov. 17, 2005

Related U.S. Application Data
(60) Provisional application No. 60/479,848, filed on Jun. 20, 2003.

(51) Int. Cl. A01N 1/02

(52) U.S. Cl. 435/2

(57) ABSTRACT

Platelet resuspension solutions, platelet storage solutions and systems for processing platelet-containing biological fluids are disclosed, wherein a platelet resuspension solution comprises an aqueous solution having a pH in the range of from about 4 to about 6: dextrose; and citrate; wherein the solution is substantially free of adenine, and wherein a platelet storage solution comprises an aqueous solution having a pH in the range of from about 6.6 to about 7.8; at least one buffer: dextrose, and citrate; wherein the solution is substantially free of adenine.

### CROSS-REFERENCE TO RELATED PATENT APPLICATIONS

This patent application claims the benefit of U.S. Provisional Patent Application No. 60/479,848, filed Jun. 20, 2003, which is incorporated by reference.

### FIELD OF THE INVENTION

This invention pertains to solutions, methods, and systems for processing platelet-containing biological fluids, more particularly, for resuspending and/or storing platelet products.

### BACKGROUND OF THE INVENTION

Blood is typically processed to separate various blood components that can be separately used. For example, a unit of donated whole blood can be processed to separate red cells, usually concentrated as packed red cells (PRC), platelets, usually concentrated as platelet concentrate (PC), and plasma. In accordance with typical processing protocols, blood is processed to form, among other fractions, a platelet-containing fluid, e.g., platelet-rich-plasma (PRP) or buffy coat, that are further processed (including centrifugation) to form the PC. Moreover, multiple units of platelets or buffy coat can be pooled before producing the final transfusion product.

In accordance with current conventional blood banking practice, PC produced in a closed system can be stored for up to 5 days before being used as a transfusion product. In some processing protocols, a platelet additive solution is added to the platelet-containing fluid (e.g., the buffy coat) and the platelets are resuspended in the additive solution before the platelets are stored, wherein most of the plasma is removed before the additive solution is added. In order to provide optimal platelet function and viability during storage, it is recommended that the platelet-containing fluid (with or without an additive solution) be maintained at a pH in the range of from 6.8 to 7.4 (European practice), or maintained at a pH of 6.2 or greater (US practice) during the storage period. It is also recommended that the platelets be stored in the presence of glucose to maintain platelet quality.

However, commercially available sterile platelet additive solutions, which have a pH in the range of 7.0-7.2, do not contain glucose, due to difficulties (e.g., glucose caramelization) encountered when steam sterilizing glucose-containing additive solutions having such a pH range. Accordingly, since plasma contains glucose, when these sterilized commercially available solutions are used, at least about 10% of the initial volume of plasma from a whole blood unit must remain with the platelets (e.g., about 30-50 ml of plasma remains from the buffy coat, or about 40-75 ml of plasma remains from the PRP, the rest of the plasma is removed) to provide sufficient glucose for the storage period. In view of the volume of additive solution added to the platelet-containing fluid, the platelets are diluted, and this can provide an increased fluid load on a patient receiving the platelets as a transfusion product.

Additionally, platelets may become activated during the processing of blood to concentrate the platelets (including during the subsequent resuspension of the platelets in the additive solution), leading to platelet aggregation and a loss of viable platelets in the transfusion product.

The present invention provides for ameliorating at least some of the disadvantages of the prior art. These and other advantages of the present invention will be apparent from the description as set forth below.

### BRIEF SUMMARY OF THE INVENTION

In one embodiment, the invention provides a platelet resuspension solution, comprising an aqueous solution having a pH in the range of from about 4 to about 6, dextrose (glucose), and citrate, wherein the solution is essentially free of adenine. In a preferred embodiment, the platelet resuspension solution comprises a sterilizable solution.

In another embodiment, the invention provides a platelet storage solution, comprising an aqueous solution having a pH in the range of from about 6.6 to about 7.8, dextrose, citrate, and a buffer, wherein the solution is essentially free of adenine. In a preferred embodiment, the buffer comprises sodium bicarbonate.

A platelet processing system according to an embodiment of the invention comprises a flexible bag and platelet resuspension solution contained in the bag.

In yet another embodiment, a platelet storage system is provided, comprising a flexible bag suitable for containing a platelet-containing solution, the bag having gas permeable side walls, and further comprising a buffering material. The buffering material can be in the bag, or in a compartment communicating with the bag.

In another embodiment, a biological fluid processing system is provided, comprising the platelet processing system, and at least one, and preferably, at least two, additional flexible bags. In a more preferred embodiment, at least one of the additional flexible bags comprises a gas permeable bag.

Methods of using the platelet additive solutions, the platelet processing system, and the platelet storage system, and the biological fluid processing systems are also provided.

### DETAILED DESCRIPTION OF THE INVENTION

In one embodiment, the invention provides a platelet resuspension solution, comprising an aqueous solution having a pH in the range of from about 4 to about 6, preferably in the range from about 5 to about 5.5, dextrose (glucose), and citrate, wherein the solution is substantially free of adenine. In a preferred embodiment, the platelet resuspension solution comprises a sterilizable solution.

In another embodiment, the invention provides a platelet storage solution, comprising an aqueous solution having a pH in the range of from about 6.6 to about 7.8, preferably in the range of from about 6.8 to about 7.4, dextrose, citrate, and a buffer, wherein the solution is essentially free of adenine. In a preferred embodiment, the buffer comprises sodium bicarbonate.

Preferred embodiments of the resuspension and/or storage solutions include acetate, more preferably, sodium acetate. Without being limited to any particular mechanism, it is believed the presence of sodium acetate in the resuspension solution and/or storage solution reduces glycolysis, thus making it easier to control of pH of the solution(s). With respect to the storage solution, this may reduce the amount of buffer utilized in the solution.

Typically, the platelet resuspension and/or platelet storage solution also includes electrolytes for ionic balance. For example, preferred embodiments of the solution(s) include at least one of, and preferably, at least two of, sodium chloride (for example, physiological saline solution), potassium chloride, and magnesium chloride, as these electrolytes are more natural to the human body (e.g., as they are more similar to those electrolytes found in plasma).

The platelet resuspension and/or platelet storage solution can also include additional components such as, for example, phosphate and/or citric acid.

Typically, the platelet resuspension and/or platelet storage solution has an osmolarity in the range of from about 260 to about 380 mOsmAL, preferably, in the range of from about 280 to about 320 mOsm/L, as measured in accordance with U.S. Pharmacopeia (USP) 24-NF19.

In accordance with an embodiment of a platelet resuspension solution according to the invention, the solution has a pH in the range of from about 4 to about 6, preferably, in the range of from about 4.6 to about 5.7, even more preferably, in the range from about 5 to about 5.7. In preferred embodiment, the platelet resuspension solution is a sterilizable solution, more preferably, a steam sterilizable solution.

In accordance with an embodiment of a platelet storage solution according to the invention, the solution has a pH in the range of from about 6.6 to about 7.8, preferably, in the range of from about 6.8 to about 7.4. Typically, at a temperature in the range of from about 22° C. to about 24° C., the solution has a pH in the range of from about 6.8 to about 7.6, and at a temperature in the range of from about 27° C. to about 39° C., the solution typically has a pH in the range of from about 7 to about 7.8.

Typical embodiments of platelet resuspension and/or platelet storage solutions include concentrations in the range of from about 10 to about 45 mM glucose, about 6 to about 10 mM sodium citrate, and 0 to about 25 mM sodium acetate. In those embodiments also including electrolytes, the solution(s) typically include at least one, and more preferably, each of the following, in the following concentration(s): in the range of from about 3 to about 7 mM potassium chloride, about 1 to about 5 mM magnesium chloride, and about 70 to about 130 mM sodium chloride. The solution(s) may also include, for example, in the range of from 0 to about 5 mM citric acid and/or in the range of about 3.5 to about 7 mM sodium phosphate (in some embodiments, in the range of about 3.5 to about 4.5 mM sodium phosphate).

Preferred embodiments of platelet resuspension and/or platelet storage solutions include concentrations in the range of from about 15 to about 30 mM glucose, about 7 to about 9 mM sodium citrate, and 8 to about 18 mM sodium acetate. In those embodiments also including electrolytes, the solution(s) typically include at least one, and more preferably, each of the following in the following concentration(s): in the range of from about 3 to about 5 mM potassium chloride, about 2 to about 4 mM magnesium chloride, and about 80 to about 120 mM sodium chloride. The solution(s) may also include, for example, citric acid having a concentration in the range of from 1 to about 4 mM and/or sodium phosphate having a concentration in the range of about 3 to about 6.5 mM.

In those embodiments wherein the platelet resuspension solution includes citric acid and sodium citrate, the mM ratio of citric acid/sodium citrate is preferably in the range of from about 0.1 to about 0.5.

With respect to a buffer, e.g., as included in the platelet storage solution, a preferred buffer is sodium bicarbonate, in a concentration in the range of from about 5 to about 20 mM, more preferably in the range of from about 8 to about 15 mM. In accordance with preferred embodiments of the invention, the buffering material is sterilized before it is utilized in forming an embodiment of the platelet storage solution or before it is added to a platelet-containing resuspension solution.

As used herein, a solution substantially free of adenine has an adenine concentration of 0.2 mM or less, preferably, 0.1 MM or less.

A platelet processing system according to an embodiment of the invention comprises a flexible bag and the platelet resuspension solution as described above contained in the bag. In some embodiments, the flexible bag is suitable for containing a platelet- and platelet resuspension-containing solution

In yet another embodiment, a platelet storage system is provided, comprising a flexible bag suitable for containing (preferably, for storing) a platelet-containing solution, the bag being a gas permeable bag, and further comprising a buffering material. In some embodiments of the system, the bag contains the buffering material, or the system further comprises a compartment communicating with the flexible bag, wherein the compartment, preferably a squeezable compartment, contains the buffering material. In an embodiment of the system, the bag contains the platelet storage solution as described above therein.

In another embodiment, a biological fluid processing system is provided, comprising the platelet processing system as described above, and at least one, and preferably, at least two, additional flexible bags. In a more preferred embodiment, at least one of the additional flexible bags comprises a gas permeable bag.

In another embodiment, a biological fluid processing system comprises the platelet processing system as described above, and an apheresis bowl, chamber, or tube, suitable for containing a platelet-containing biological fluid. The system can include at least one additional container, e.g., a flexible, gas permeable bag.

In accordance with the invention, platelets can be effectively resuspended in the platelet resuspension solution (in some embodiments, resuspended in the platelet storage solution) while reducing activation and the formation of aggregates, thus increasing the yield of valuable platelets for transfusion. Moreover, the platelets can be stored while maintaining high platelet quality, thus providing a high yield of useable platelets for transfusion.

Furthermore, the platelets can be prepared and stored while requiring less plasma in the stored product than is conventionally used, thus increasing the yield for valuable plasma for other uses. For example, while conventional methods require utilizing about at least about 10% of the initial volume of plasma in preparing platelet products for storage, platelet products suitable for storage can be prepared in accordance with embodiments of the invention utilizing about 50% or less plasma than that used in the conventional methods, typically, about 40% or less, and in some embodiments, about 30% or less.

For example, in a conventional method wherein about 30 to about 35 ml of plasma from buffy coat is used for preparing a platelet product for storage, embodiments of the invention can utilize about 10 to about 15 ml of plasma. In another conventional method wherein about 50 to about 60 ml of plasma from platelet-rich-plasma (PRP) is used for preparing a platelet product for storage, embodiments of the invention can utilize about 5 to about 15 ml of plasma

There are other advantages associated with the use of less plasma For example, platelet products can be prepared having less total volume, thus decreasing the load on the patient receiving the transfusion product. Moreover, the platelet products can be prepared containing a greater number of platelets without requiring a great increase in the total volume of the product to obtain the additional platelets. Yet another advantage is the additive solution according to the invention adds less fluid volume to the platelet product than conventional additives. For example, while conventional additives would add about 300 ml of additive solution to, for example, 4 pooled buffy coats, preferred embodiments of the invention would add about 200 ml of the inventive additive solution or less. With respect to non-pooled platelet products, e.g., single random donor units, units can be prepared having, if desired, a total volume of about 40 to about 50 ml.

Typically, the platelet resuspension solution and/or platelet storage solution is essentially free of photoactive agents (e.g., photosensitizers for use in inactivating microorganisms). However, the solution(s) can be used with photosensitizers, and, in microorganism inactivation protocols such as viral inactivation protocols, the presence of less plasma allows less inactivation agent (e.g., psoralens such as methylene blue), or a lower dose of the inactivation agent (e.g., UV light) to be utilized during the protocol. Thus, there can be less inactivation agent to be subsequently removed and/or platelet damage can be reduced.

In accordance with preferred embodiments of a method according to the invention, a platelet-containing biological fluid (e.g., comprising apheresis platelets, platelets obtained from platelet-rich-plasma or platelets obtained from pooled buffy coats), is combined with a platelet resuspension solution to form a platelet-containing platelet resuspension solution, and the platelets are resuspended in the solution. In some embodiments, e.g., some apheresis systems, the platelet resuspension solution comprises an elutriation solution. The platelet-containing platelet resuspension solution is further processed, which includes adding a buffer, and the buffered platelet-containing fluid is stored in a container (preferably a gas permeable flexible bag) for a desired period of time before further use, e.g., as a transfusion product that is administered to a patient.

Embodiments of the method can include pooling two or more volumes of platelet-containing fluid (e.g., two or more units of buffy coat), and mixing pooled platelet-containing fluid with a platelet-resuspension solution to provide a pooled platelet-containing platelet resuspension solution. For example, in one embodiment, 4-6 units of whole blood are each processed to provide sedimented red cells, buffy coat, and platelet-poor-plasma (PPP), and the components are separated. As explained above, in preferred embodiments, a greater volume of PPP can be removed when separating the PPP from the buffy coat, as the PPP is not needed to provide glucose. The buffy coats (the platelet-containing fluid) from each unit of whole blood are pooled, the pooled platelet-containing fluid is mixed with a platelet resuspension solution, and the platelets are re-suspended in the resuspension solution. The fluid is further processed so that platelets can be prepared for storage. For example, further processing includes adding a buffering material (typically after centrifugation and separation of biological fluid components), and the resultant supernatant pooled platelet-containing fluid can subsequently be separated from the sedimented red and white blood cells. The separated pooled platelet-containing fluid (containing the buffered resuspension solution, thus forming an embodiment of the. storage solution), can be stored as described above before further use.

In one embodiment that does not include pooling, a unit of whole blood is centrifuged to form supernatant platelet-rich-plasma (PRP) and sedimented packed red blood cells, and the PRP is expressed to, for example, a container such as a blood bag, e.g., a plasticized satellite bag. The PRP is subsequently centrifuged to concentrate the platelets at the bottom of the bag, and the supernatant plasma is expressed from the bag. As explained above, in preferred embodiments, a greater volume of plasma can be removed when separating the plasma from the concentrated platelets, as this plasma is not needed to provide glucose. After the plasma is removed, platelet resuspension solution (e.g., about 40 to about 50 ml) is added to the bag, and the platelets are resuspended in the resuspension solution. In some embodiments, the platelets are resuspended in the resuspension solution after the platelets have rested unagitated for a period of time, for example, about 1-2 hours. Subsequently, a buffer is added, thus forming an embodiment of the storage solution, and the buffered, platelet-containing solution can be stored as described above before further use.

In another embodiment, after the PRP is centrifuged and the supernatant plasma is removed as described above, non-platelet containing buffered platelet storage solution (e.g., about 40 to about 50 ml of the solution) is added to the bag, and the platelets are (in some embodiments, after the platelets have rested unagitated for a period of time) resuspended in the buffered storage solution, and can be stored in the solution.

In accordance with another embodiment that does not include pooling, e.g., wherein the biological fluid is processed in an apheresis system, concentrated platelets are prepared using the apheresis system (e.g., including, but not limited to, an apheresis system such as a Baxter Fenwall Amicus® Separator or a Baxter Fenwall CS 3000 plus) in a container (e.g., a collection chamber), and an embodiment of the resuspension solution (e.g., about 100 to about 300 ml for single product platelets) is added to the concentrated platelet fluid, preferably, without adding plasma to the concentrated platelet fluid. The platelets are resuspended in the resuspension solution (in some embodiments, the platelets are resuspended after the platelets have rested unagitated for a period of time). Subsequently, a buffer is added, thus forming the storage solution, and the buffered, platelet-containing solution can be stored as described above before further use.

In another embodiment, after concentrated platelets are prepared using the apheresis system in a container (e.g., a collection chamber) as described above, an embodiment of the non-platelet containing buffered storage solution (e.g., about 100 to about 300 ml of the solution for single product platelets) is added to the concentrated platelet fluid, preferably, without adding plasma to the concentrated platelet fluid. The platelets are (in some embodiments, after the platelets have rested unagitated for a period of time) resuspended in the buffered storage solution, and can be stored in the solution.

In preferred embodiments of the invention, leukocytes are depleted from the platelets. For example, a platelet-containing fluid, that can be prepared, for example, in an apheresis system or from a donated unit of whole blood, e.g., PRP, buffy coat, or the supernatant layer comprising platelets formed after pooled buffy coats are centrifuged, can be passed through a leukocyte depletion filter (preferably a leukocyte depletion filter including a leukocyte depletion- and red cell barrier-medium) to provide leukocyte-depleted platelets. A platelet-containing fluid can be leukocyte-depleted before forming the platelet-containing platelet resuspension solution, or before forming a platelet-containing storage solution, or after forming the platelet-containing platelet resuspension solution, but before adding a buffer. Preferably, a platelet-containing fluid is filtered before a buffer is added to the fluid.

In those embodiments wherein a platelet-containing fluid is combined with a buffered platelet storage solution (e.g., some embodiments wherein PRP is centrifuged and the supernatant plasma is removed, and the buffered platelet storage solution is mixed with the concentrated platelets), platelets are preferably filtered before a platelet-containing fluid is combined with a buffered platelet storage solution.

A typical embodiment of the method further comprises storing the buffered platelet-containing solution (preferably, wherein the platelets have been leukocyte-depleted) in the container for at least 2 days, preferably, at least 5 days, and in some embodiments, at least 7 days. Preferably, the pH of the fluid is maintained within the range of about 6.8 to about 7.4 during the storage period, more preferably, wherein the fluid is stored in a gas permeable flexible bag.

If desired, a preferred embodiment of the method further comprises administering platelets to a patient.

In accordance with current U.S. practice, platelet-containing biological fluids prepared in closed systems (with or without additive solutions) can be stored for 5 days before use, e.g., as transfusion products, and platelets processed according to the invention can be stored for that period of time. However, platelets produced in accordance with embodiments of the invention can remain viable for longer periods of time, e.g., they remain viable after 7 days of storage, after 10 days of storage, and even after 14 days of storage. Accordingly, should the regulations in the U.S., or any other country be changed, embodiments of the invention allow for platelet storage for longer than 5 days, e.g., up to about 7 days or more, or 10 days, or even, 14 days, or more.

The viability of the platelets can be determined by a variety of methods known in the art. Typically, in determining viability, at least one, and more preferably, two or more, of the following are evaluated: platelet count, pH, pO₂, pCO₂, bicarbonate, streaming (or swirling), hypotonic shock response (% HSR), extent of shape change (% ESC), % discs (platelet morphology), CD62 level (p-selectin), plasma glucose, plasma lactate, ATP level, and in vivo radiolabeling studies.

A variety of containers, preferably flexible blood bags, are suitable for use in accordance with the invention. The containers should be sterilizable in accordance with conventional protocols, e.g., at least one of steam sterilization, gamma sterilization, and ethylene oxide sterilization.

In some embodiments, the platelets can be resuspended in the platelet resuspension solution in one container (for example, in the collection chamber of an apheresis system, or in a flexible blood bag), and the buffered platelet-containing solution can be formed in another container, for example. Typically, the container utilized for storing the platelets (that may be the container in which the buffered platelet storage solution is formed) is a gas permeable container, e.g., a container allowing suitable gas transmission into and/or out of the interior volume of the container.

Examples of suitable flexible containers, wherein the containers are gas permeable, include, but are not limited to, polyolefin elastomer bags as described in International Publication No. WO 02065976, bags prepared from a film comprising ultra high molecular weight plasticized PVC as described in U.S. Pat. No. 5,721,024, and bags prepared from a film comprising plasticized PVC as described in U.S. Pat. No. 4,280,497.

Preferably, the polymeric film used in manufacturing gas permeable bags has a 22° C. room air oxygen transmission of about 12 μmoles or greater O₂/hr/350 cm² film surface area. In some embodiments, the 22° C. room air oxygen transmission is 15 μmoles or greater O₂/hr/350 cm² film surface area, preferably, about 18 μmoles or greater O₂/hr/350 cm² film surface area, and even more preferably, about 20 μmoles or greater O₂/hr/350 cm² film surface area.

The containers used in accordance with some embodiments are also resilient to temperature fluctuations, e.g., they can withstand low temperatures during freezing, e.g., when processing plasma.

In some embodiments, at least one container, e.g., wherein the container comprises a gas permeable container, is free of, or essentially free of, plasticizers such as di (2-ethylhexyl) phthalate (DEHP), tri (2-ethylhexyl) trimellitate (TOTM), and citrate ester plasticizers such as n-butryl tri-n-hexyl citrate (BTHC). However, one or more containers (e.g., the polymeric film) can include modifiers and/or additives such as, for example, at least one of an antistatic, antiblock, a stabilizer, and antioxidant, e.g., for use in processing the film for making the containers.

As described in International Publication No. WO 02065976, a resin is used in producing the polymeric film used in manufacturing a polyolefin elastomer bag and the resin comprises at least one copolymer comprising ethylene and an acrylate, preferably comprising ethylene and an alkyl acrylate. The resin can comprise a plurality of copolymers, e.g., a blend comprising a first copolymer comprising ethylene and a first alkyl acrylate, and a second copolymer comprising ethylene and a second alkyl acrylate.

In some embodiments, the copolymer comprises ethylene and at least about 20 weight percent alkyl acrylate based upon the combined weight of the ethylene and the alkyl acrylate. For example, the copolymer can comprise ethylene and at least about 22 weight percent alkyl acrylate, or ethylene and at least about 24 weight percent alkyl acrylate. The term “alkyl” herein refers to an alkyl group having from 1 to about 10 carbon atoms, preferably from 1 to about 6 carbon atoms, and more preferably from 1 to about 4 carbon atoms. In even more preferred embodiments, the alkyl acrylate is methyl acrylate or butyl acrylate. For example, the resin can comprise a copolymer comprising ethylene, and at least about 20 wt. % methyl acrylate or at least about 20 wt. % butyl acrylate. In other embodiments, the resin comprises a copolymer comprising ethylene, and at least about 22 wt. % methyl acrylate or at least about 22 wt. % butyl acrylate, or ethylene and at least about 24 wt. % methyl acrylate or at least about 24 wt. % butyl acrylate.

Suitable resins include, for example, resins commercially available from, for example, Eastman Chemical Company (Kingsport, Tenn.), Atofina Chemicals, Inc. (Philadelphia, Pa.) and Dupont (Wilmington, Del.). For example, a variety of resins commercially available from Eastmnan Chemical Company referred to as EMAC® (including EMAC+®), EBAC® (including EBAC+®), and EMAC/EBAC® are suitable. Illustrative examples of such resins are ethylene butyl acrylate copolymer (EBAC) resin, e.g., EBAC SP1802 and SP1903 specialty copolymers, and ethylene methyl acrylate copolymer (EMAC) resin, e.g., EMAC SP1305, SP1307, SP1330, SP1400, SP2202, SP2207, SP2220, SP2260 and SP2268, specialty copolymers. Illustrative suitable resins commercially available from Atofina Chemicals, Inc., include, for example, those resins referred to as LOTRYL™ resins (e.g., LOTRYL™ EBA and LOTRYL™ EMA) and illustrative suitable resins commercially available from DuPont include, for example, those resins referred to as ELVALOYT™ resins (e.g., ELALOY™ AC).

As described in U.S. Pat. No. 5,721,024, a flexible container can comprise a polyvinyl chloride (PVC) film manufactured from a polyvinyl chloride compound, said compound comprising an ultra high molecular weight polyvinyl chloride resin having an inherent viscosity ranging from about 1.25 to about 2.00, as measured by ASTM D-1243; and about 43 weight percent or more (typically, in the range of from about 43 to about 57 weight percent) of a plasticizer. Preferably, the plasticizer is one from the group of plasticizers consisting of: tri (2-ethylhexyl) trimellitate; di-(2-ethylhexyl) phthalate; acetyl tri-n-butyl citrate; n-butyryl tri-n-hexyl citrate; acetyl tri-n-octyl citrate; and acetyl tri-n-decyl citrate.

The bags according to the invention can have any suitable size, shape, internal volume and/or thickness. The bags can be made from the polymeric film and resin described herein using conventional techniques known and used in the industry. Illustratively, the bag can be arranged from a single sheet of sheet of film (e.g., folded over at the end where the ports are arranged and sealed around the other edges), two sheets of film, from a collapsed blown bubble of film (sometimes referred to as “lay flat tubing”), and the like. The bags are typically extruded, but can be blow molded or formed by other appropriate methods known in the art.

The preferred wall thickness of gas permeable containers for storing platelet-containing fluids using the polymeric film can be in the conventional range ofqabout 0.005 to about 0.025 inch (about 0.13 to about 0.64 mm), preferably about 0.010 inch to about 0.018 inch (about 0.25 to about 0.46 mm), with about 0.012 to about 0.015 inch (about 0.30 to about 0.38 mm) being most preferred. This wall thickness results in containers having sufficient tensile strength to withstand conventional use in the collection and processing of blood and blood components.

In typical embodiments of gas permeable containers according to the invention, each side wall is a single layer of film.

In a preferred embodiment, the gas permeable bag is configured to allow at least one material (e.g., a dry material, or a liquid), more preferably, a buffering material, even more preferably a buffer that can be subjected to sterilization conditions without substantial degradation, to be mixed with a fluid, e.g., the platelet-containing platelet resuspension solution mixture, or a non-platelet containing resuspension solution, in the bag. The material can be present (e.g., as a tablet) in the interior volume of the bag, e.g., so that the platelet-containing platelet resuspension solution mixture contacts the material when the solution mixture is passed into the bag.

Alternatively, gas permeable container can include at least two compartments, e.g., a larger compartment for containing a platelet-containing platelet resuspension solution mixture, and at least one smaller compartment containing the material wherein the bag is arranged to allow the material in the smaller compartment to be passed to the larger compartment. In yet another embodiment, an additional container is arranged so that material from the additional container can be passed into the container containing the platelet-containing platelet resuspension solution mixture. Typically, the smaller compartment or the additional container is adapted to contain a buffer. Between the smaller compartment and the larger compartment, and between the additional container and the gas permeable container, is a closure means, preferably an externally manipulable closure means (in some embodiments, an in-line frangible valve), that allows the buffer to be mixed with the platelet-containing platelet resuspension solution mixture when desired. In one embodiment, the gas permeable bag includes at least two compartments as disclosed in U.S. Pat. Nos. 4,902,287 and 4,994,057, wherein at least one smaller compartment is squeezable, i.e., the smaller compartment comprises a resilient material that causes the compartment to generally return to its original shape after it has been deformed by external pressure on the compartment walls.

While a variety of buffers are suitable for use in accordance with the invention, a preferred material is sodium bicarbonate. The buffering material is preferably in dry form, e.g., in powder or tablet form. As noted above, preferably, the buffer can be subjected to sterilization conditions without substantial degradation.

A variety of leukocyte depletion filters comprising leukocyte media are suitable for use in according to the invention. Alternatively, or additionally, in some embodiments, the leukocyte depletion filter comprises a red cell barrier medium, or a combined red cell barrier leukocyte depletion medium. Suitable filters and media include, but are not limited to, those disclosed in U.S. Pat. Nos. 4,880,548, 5,100,564, 5,152,905, 5,472,621 and 5,670,060.

The following definitions are used in accordance with the invention.

Biological Fluid. A biological fluid includes any treated or untreated fluid associated with living organisms, particularly blood, including whole blood, warm or cold blood, and stored or fresh blood; treated blood, such as-blood diluted with at least one physiological solution, including but not limited to saline, nutrient, and/or anticoagulant solutions; blood components, such as platelet concentrate (PC), platelet-rich plasma (PRP), platelet-poor plasma (PPP), platelet-free plasma, plasma, fresh frozen plasma (FFP), components obtained from plasma, packed red cells (PRC), transition zone material or buffy coat (BC); blood products derived from blood or a blood component or derived from bone marrow; stem cells; red cells separated from plasma and resuspended in physiological fluid or a cryoprotective fluid; and platelets separated from plasma and resuspended in physiological fluid or a cryoprotective fluid. The biological fluid may have been treated to remove some of the leukocytes before being processed according to the invention. As used herein, blood product or biological fluid refers to the components described above, and to similar blood products or biological fluids obtained by other means and with similar properties.

A “unit” is the quantity of biological fluid from a donor or derived from one unit of whole blood. It may also refer to the quantity drawn during a single donation. Typically, the volume of a unit varies, the amount differing from patient to patient and from donation to donation. Multiple units of some blood components, particularly platelets and buffy coat, may be pooled or combined, typically by combining four or more units.

As used herein, the term “closed” refers to a system that allows the collection and processing (and, if desired, the manipulation, e.g., separation of portions, separation into components, filtration, storage, and preservation) of biological fluid, e.g., donor blood, blood samples, and/or blood components, without the need to compromise the sterile integrity of the system. A closed system can be as originally made, or result from the connection of system components using what are known as “sterile docking” devices. Illustrative sterile docking devices are disclosed in U.S. Pat. Nos. 4,507,119, 4,737,214, and 4,913,756.

Sterilizable preferably means the capability of being subjected to temperatures of at least about 114° C. for at least about 30 minutes (or exposed to at least about 2.5 megarads of gamma radiation) without significant degradation of a given product. In the case of dry or liquid compounds, chemicals, or components, such compounds, chemicals and components preferably retain at least about 75% by weight of their initial pre-sterilization chemical identity and utility after having been subjected to the above sterilization conditions.

The following examples further illustrate the invention but, of course, should not be construed as in any way limiting its scope.

### EXAMPLE 1

This example describes the preparation of an embodiment of the platelet resuspension solution, as well as the preparation of an embodiment of a platelet processing system.

A 200 mL platelet resuspension solution with a pH of 5.3 and a calculated osmolarity of 340 mOsm/L is prepared having the following:

| | g/L | mM |
|---|---|---|
| Dextrose H₂O | 5.94 | 30 |
| Potassium Chloride | 0.37 | 5 |
| Magnesium Chloride 6H₂O | 0.61 | 3 |
| Sodium Chloride | 6.43 | 110 |
| Citric acid | 0.48 | 2.5 |
| Sodium Citrate 2H₂0 | 2.21 | 7.5 |
| Sodium Phosphate | 0.55 | 4 |
| monobasic H₂O | | |
| Sodium Acetate 3H₂O | 2.04 | 15 |

The solution is placed in a plasticized PVC bag, and steam sterilized to provide a platelet processing system.

### EXAMPLE 2

This example described the preparation of an embodiment of a platelet storage system according to the invention.

A gas permeable polyolefin elastomer platelet storage bag is prepared as generally described in International Publication No. WO 02065976. The bag contains a buffering material, a sodium bicarbonate tablet (168 mg).

The bag, including the tablet, is gamma sterilized.

### EXAMPLE 3

This example describes the resuspension of a platelet-containing solution using embodiments of a platelet storage solution, a platelet processing system, and a platelet storage system, according to the invention.

Four units of anticoagulated whole blood are each processed in top/bottom bags to produce supernatant platelet-poor-plasma (PPP), buffy coat (BC), and sedimented packed red blood cells (PRC), wherein the PPP and PRC are separated from the BC. In separating the PPP from the BC, about 10-15 mL of PPP remains with the BC.

The four top/bottom bags each containing BC therein are sterile connected together in series. The platelet processing system as described in Example 1, containing the 200 mL of resuspension solution, is sterile connected to the top port of the first (top) top/bottom bag in the series. A second plasticized PVC bag is sterile connected to the bottom port of the last (bottom) top/bottom bag in the series.

A filter including a leukocyte depletion and red cell barrier medium as described in U.S. Pat. No. 5,670,060 is obtained, and sterile connected between the second plasticized PVC bag and the platelet storage system described in Example 2.

The four units of BC connected in series are drained from each bag and pooled into the plasticized PVC bag, and the sterile resuspension solution is passed through the top port of the first bag and through the other 3 top/bottom bags into the PVC bag.

The conduit between the bottom port of the last top/bottom bag and the second PVC bag is heat-sealed and cut.

The platelet- and platelet resuspension solution-containing fluid is centrifuged to provide a supernatant layer including platelets, and a sediment layer including red blood cells.

### EXAMPLE 4

This example describes the leukocyte-depletion and storage of a platelet-containing solution using an embodiment of a platelet storage system, according to the invention.

The supernatant layer described in Example 3 is passed from the second PVC bag, through the filter, into the platelet storage system, wherein the supernatant layer contacts the sodium bicarbonate tablet in the polyolefin elastomer bag. The concentration of sodium bicarbonate in the solution is 10 mM.

The conduit between the outlet of the filter and the port of the platelet storage system is heat-sealed and cut.

The gas permeable bag including the buffered solution therein is placed on a horizontal shaker in a 20-24° C. controlled environmental chamber. Platelet quality is measured on days 2, 5, 7, and 9 of storage.

Additionally, the platelet concentration in the container is determined to be greater than 1.4 ×10⁹ platelets/mL.

The pH is well maintained at a pH of 7.2 to 7.4 during the storage period, and the platelets remain viable as reflected by analysis of the ektent of shape change (ESC) and the hypotonic shock response (HSR).

Examples 3 and 4 show platelets can be resuspended and stored in accordance with embodiments of the invention, and platelet viability (at a concentration of over 1.4×10⁹ platelets/mL) can be maintained for at least a 9 day storage period.

### EXAMPLE 5

This example describes the preparation of an embodiment of the platelet storage solution.

A 50 mL solution with a pH of 5.3 and a calculated osmolarity of 340 mOsm/L is prepared having the following:

| | g/L | mM |
|---|---|---|
| Dextrose H₂O | 5.94 | 30 |
| Potassium Chloride | 0.37 | 5 |
| Magnesium Chloride 6H₂O | 0.61 | 3 |
| Sodium Chloride | 6.43 | 110 |
| Citric acid | 0.48 | 2.5 |
| Sodium Citrate 2H₂0 | 2.21 | 7.5 |
| Sodium Phosphate | 0.55 | 4 |
| monobasic H₂O | | |
| Sodium Acetate 3H₂O | 2.04 | 15 |

A plasticized PVC bag is obtained, and a squeezable compartment is formed from plasticized PVC and attached to the bag via a conduit as generally described in U.S. Pat. No. 4,902,287. The squeezable compartment includes sodium bicarbonate powder (168 mg). The conduit includes an in-line frangible valve. The plasticized PVC bag is also part of a closed multiple bag blood processing set including a collection bag, a plurality of satellite bags, and a filter comprising a leukocyte depletion and red cell barrier medium as described in U.S. Pat. No. 5,152,905.

The solution is placed in the plasticized PVC bag, and the solution, as well as the sodium bicarbonate in the squeezable container, are steam sterilized.

The in-line frangible valve is manipulated to open the flow path in the conduit between the PVC bag and the squeezable container. The PVC bag is inverted, and the squeezable compartment is squeezed several times to pass buffer into the bag and draw liquid into the compartment.

The resultant buffered platelet storage solution has a pH of 7.3, and a sodium bicarbonate concentration of 10 mM.

### EXAMPLE 6

This example describes the resuspension of a platelet-containing solution in an embodiment of the buffered platelet storage solution according to the invention.

A unit of anticoagulated whole blood is processed in a plasticized collection bag (which is one of the bags in the multiple bag set described in Example 5) to provide a supernatant layer comprising platelet-rich-plasma (PRP) and a sediment layer comprising packed red blood cells. The PRP is passed through the filter comprising a leukocyte depletion and red cell barrier medium into a gas permeable plasticized PVC satellite bag (wherein the gas permeable bag is described in U.S. Pat. No. 4,280,497) to provide leukocyte-depleted PRP in the satellite bag.

The leukocyte-depleted PRP is subjected to hard spin centrifugation, to provide concentrated platelets in the bottom of the satellite bag, and platelet-poor-plasma (PPP). About 250 ml of PPP is expressed from the satellite bag, leaving about 5 ml of plasma in the bag with the concentrated platelets. The 50 ml of buffered platelet storage is passed from the plasticized PVC bag described in Example 5 into the satellite bag, and, after a rest period of about 1.5 hours, the platelets are resuspended in the platelet storage solution wherein the gas permeable satellite bag is placed on a horizontal shaker in a 20-24 ° C. controlled environmental chamber.

Platelet quality is measured on days 2, 5, 7, and 9 of storage.

The pH is well maintained at a pH of 7.2 to 7.4 during the storage period, and the platelets remain viable as reflected by analysis of the extent of shape change (ESC) and the hypotonic shock response (HSR).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors, for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A platelet resuspension solution comprising:
an aqueous solution having a pH in the range of from about 4 to about 6;
dextrose; and
citrate;
wherein the solution is substantially free of adenine.

2. The solution of claim 1, further comprising sodium acetate.

3. The solution of claim 1, comprising a sterilizable solution.

4. The solution of claim 1, comprising a steam sterilizable solution.

5. The solution of claim 2, further comprising at least one electrolyte selected from potassium chloride, magnesium chloride, and sodium chloride.

6. The solution of claim 1, further comprising citric acid and/or sodium phosphate.

7. The solution of claim 1, having a pH in the range of from about 5 to about 5.7.

8. (canceled)

9. The system of claim 24, wherein the solution further comprises at least one electrolyte selected from potassium chloride, magnesium chloride, and sodium chloride.

10. The system of claim 24, wherein the solution further comprises citric acid and/or sodium phosphate.

11. The system of claim 24, wherein the solution further comprises having a pH in the range of from about 6.8 to about 7.4.

12. The system of claim 24, wherein the solution further comprises at least one buffer comprises sodium bicarbonate.

13. The system of claim 24, wherein the solution further comprises platelets contained therein.

14. A platelet processing system comprising:
a flexible bag; and,
an amount of the platelet resuspension solution of claim 1, contained in the bag.

15. The system of claim 14, comprising at least about 50 cc of platelet resuspension solution.

16. The system of claim 14, comprising at least about 200 cc of platelet resuspension solution.

17. The system of claim 14, comprising a sterilizable system.

18. The system of claim 17, comprising a steam sterilizable system.

19. The system of claim 14, further comprising a buffering material, separated from the platelet resuspension solution.

20. The system of claim 19, wherein the buffering material comprises sodium bicarbonate.

21. The system of claim 20, wherein the sodium bicarbonate is in solid form.

22. The system of claim 21, wherein the sodium bicarbonate is in the bag.

23. The system of claim 19, comprising a sterilized buffering material.

24. A platelet storage system comprising:
a gas permeable flexible bag;
an amount of the platelet storage solution contained in the bag, the solution comprising an aqueous solution having a pH in the range of from about 6.6 to about 7.8;
at least one buffer:
dextrose; and
citrate;
wherein the solution is substantially free of adenine, and
a leukocyte depletion filter assembly in fluid communication with the bag.

25. The system of claim 24, comprising at least about 50 cc of platelet storage solution.

26. The system of claim 24, comprising at least about 200 cc of platelet storage solution.

27. The system of claim 24, wherein the platelet storage solution further comprises platelets contained therein.

28. The system of claim 24, wherein the bag comprises side walls comprising a film manufactured from a copolymer comprising ethylene and an acrylate.

29. The system of claim 24, wherein the bag comprises side walls comprising a film manufactured from plasticized ultra high molecular weight PVC.

30. The system of claim 24, having >1.4×10⁹ platelets/mL in the bag.

31. The system of claim 14, further comprising a leukocyte depletion filter assembly in fluid communication with the bag.

32. A method of processing a biological fluid comprising:
combining a platelet-containing biological fluid with the platelet resuspension solution of any one of claim 1, to provide a platelet- and platelet resuspension solution-containing fluid.

33. The method of claim 32, including re-suspending the platelets in the platelet resuspension solution.

34. The method of claim 33, including re-suspending the platelets in the solution for a period of about 15 minutes to about 10 hours.

35. The method of claim 33, including re-suspending the platelets in the solution for a period of about 30 minutes to about 6 hours.

36. The method of claim 32, further comprising adding a buffer to the fluid to provide a buffered platelet-containing fluid; and,
storing the buffered platelet-containing fluid in a gas permeable flexible bag for at least about 24 hours while maintaining the pH of the buffered fluid.

37. The method of claim 36, wherein the buffered platelet-containing fluid is a leukocyte-depleted fluid.

38. A method of processing a biological fluid comprising:
obtaining a platelet-containing solution comprising leukocyte-depleted platelets and an aqueous solution having a pH in the range of from about 6.6 to about 7.8;
at least one buffer;
dextrose; and
citrate;
wherein the solution is substantially free of adenine; and
storing the solution in a gas permeable flexible bag for at least about 24 hours.

39. A method of processing a biological fluid comprising:
combining a platelet-containing biological fluid with the platelet storage solution of any claim 8 to provide a buffered platelet- and platelet storage solution-containing fluid.

40. The method of claim 39, wherein the buffered platelet- and platelet storage solution-containing fluid is a leukocyte-depleted fluid.
